(19) United States
(12) Patent Application Publication
(10) Pub. No.: US 2024/0139719 A1
(43) Pub. Date: May 2, 2024

(54) CATALYST FOR METHANE SYNTHESIS AND METHOD OF MANUFACTURING THE SAME

(71) Applicant: HONDA MOTOR CO., LTD., Tokyo (JP)

(72) Inventors: Hideaki YONEDA, WAKO-SHI (JP); Kazuki YANAGISAWA, WAKO-SHI (JP); Misato MAKI, WAKO-SHI (JP); Masahiro MOHRI, WAKO-SHI (JP); Jumpei YOSHIDA, WAKO-SHI (JP); Noritatsu TSUBAKI, TOYAMA-SHI (JP)

(21) Appl. No.: 18/382,724

(22) Filed: Oct. 23, 2023

(30) Foreign Application Priority Data
Oct. 26, 2022 (JP) 2022-171068

(51) Int. Cl. B01J 27/236; B01J 23/755; B01J 23/86; B01J 37/04; B01J 37/06; B01J 37/08; C01G 53/00; C07C 1/04

(52) U.S. Cl. CPC B01J 27/236; B01J 23/755; B01J 23/866; B01J 37/04; B01J 37/06; B01J 37/08; C01G 53/006; C07C 1/043; B01J 35/1019; C01P 2002/22; C01P 2002/72; C01P 2006/12; C01P 2006/14; C01P 2006/16

(57) ABSTRACT

A catalyst for methane synthesis is made up from layered double hydroxides represented by the following general formula (1).

[M²⁺₁₋ₓM³⁺ₓ(OH)₂]^{x+}[Aⁿ⁻_{x/n}·yH₂O] (1)

In formula (1), M²⁺ is Ni²⁺ and M³⁺ is Al³⁺ or Cr³⁺. Further, Aⁿ⁻ is CO₃²⁻. Furthermore, the term x lies within a range of 0.19 to 0.34 (0.19≤x≤0.34), and y is 0 or a positive integer.

### CROSS-REFERENCE TO RELATED APPLICATIONS

This application is based upon and claims the benefit of priority from Japanese Patent Application No. 2022-171068 filed on Oct. 26, 2022, the contents of which are incorporated herein by reference.

### BACKGROUND OF THE INVENTION

### Field of the Invention

The present invention relates to a catalyst for methane synthesis which is used in a synthesis reaction for obtaining methane, as well as to a method of manufacturing the same.

### Description of the Related Art

Heretofore, efforts aimed at mitigating climate change or reducing the impact of the climate change have been continued, and toward the realization thereof, research and development in relation to reducing carbon dioxide have been carried out. As a part of such efforts, attempts have been made to subject carbon dioxide and water to electrolysis and thereby obtain carbon monoxide and hydrogen, and furthermore, to synthesize methane from carbon monoxide and hydrogen. A chemical reaction formula for obtaining methane from carbon monoxide and hydrogen is shown below.

CO+3H₂->CH₄+H₂O

The above chemical reaction is promoted by a catalyst. For example, in JP 2015-502247 A, a catalyst containing a MgAl₂O₄ phase and a MgNiO₂ phase has been proposed. Further, in JP 2017-001016 A, a catalyst is exemplified having Ni, Ru, Fe, Co, V, Nb, Ta, Cr, Mo, W, Mn, Tc, or Re and alloys thereof as an active metal, and an oxide, nitride, or carbide carrier comprising at least one of Al, V, Ti, Zr, Si, Mg, and Ce. According to JP 2015-502247 A and JP 2017-001016 A, these catalysts function as CO selective methanation catalysts that selectively methanize carbon monoxide.

Apart from the above, attempts have also been made to synthesize methanol from carbon dioxide. In JP 2020-510599 A, a method is disclosed for producing layered double hydroxides for use as a catalyst in such a synthesizing process.

### SUMMARY OF THE INVENTION

A catalyst for obtaining methane is required to have an ability to cause as large an amount of carbon monoxide as possible to participate in the reaction. In this instance, in the reaction for obtaining methane from carbon monoxide and hydrogen, carbon dioxide is generated as a by-product. Further, hydrocarbon compounds other than methane are also generated as a by-product. The carbon dioxide and the hydrocarbon compounds reduce the purity of the methane. Therefore, the catalyst is also required to have an ability to preferentially promote the synthesis reaction of methane.

The present invention has the object of solving the aforementioned problems.

According to one embodiment of the present invention, there is provided a catalyst for methane synthesis that is made up from layered double hydroxides represented by the following general formula (1), and that promotes a synthesis reaction for obtaining methane,

[M²⁺₁₋ₓM³⁺ₓ(OH)₂]^{x+}[Aⁿ⁻_{x/n}·yH₂O]^{x-} (1)

wherein, in formula (1), M²⁺ and Aⁿ⁻ are Ni²⁺ and CO₃²⁻, respectively, and M³⁺ is Al³⁺ or Cr³⁺, where 0.19≤x≤0.34, and y is 0 or a positive integer.

According to another aspect of the present invention, there is provided a method of manufacturing a catalyst for methane synthesis that is made up from layered double hydroxides represented by the following general formula (1), and that promotes a synthesis reaction for obtaining methane,

[M²⁺₁₋ₓM³⁺ₓ(OH)₂]^{x+}[Aⁿ⁻_{x/n}·yH₂O]^{x-} (1)

wherein, in formula (1), M²⁺ and Aⁿ⁻ are Ni²⁺ and CO₃²⁻, respectively, and M³⁺ is Al³⁺ or Cr³⁺, where 0.19≤x≤0.34, and y is 0 or a positive integer, the method comprising a mixing step of dissolving nickel nitrate, chromium nitrate or aluminum nitrate, and urea in water and thereby obtaining a mixed solution, a heating step of heating and pressurizing the mixed solution and thereby obtaining a generated product, a cleaning step of cleaning the generated product, and a firing step of firing the generated product that has been cleaned and thereby obtaining the layered double hydroxides.

The catalyst for methane synthesis which is made up from the above-described layered double hydroxides is superior in terms of catalytic activity. More specifically, by using such a catalyst, it is possible to convert a large amount of carbon monoxide into methane within a short period of time. In addition, the generation of by-products (carbon dioxide or organic compounds) other than methane is suppressed. In this manner, such a catalyst is superior in terms of its ability to cause a large amount of carbon monoxide to participate in the reaction, and is also superior in terms of its ability to preferentially promote the synthesis reaction of methane.

Further, such a catalyst also exhibits superior resistance to water. The carbon monoxide and the hydrogen generated through electrolysis contain moisture. Further, water is generated in the methane synthesis reaction between carbon monoxide and hydrogen. Even under such an environment in which a comparatively large amount of moisture is present, the catalyst maintains its catalytic activity. Therefore, it is possible to eliminate a dehumidifier from being provided between an electrolysis device and a methane synthesis device.

Consequently, it is possible to simplify the methane synthesis system and to reduce the cost of investment in equipment. In addition, since it is possible to eliminate incidental equipment and the like provided in the dehumidifier, the running cost when continuously operating the methane synthesis system is reduced.

Furthermore, according to the aforementioned manufacturing method, layered double hydroxides (catalysts) with a small particle size and a narrow particle size distribution width can be easily obtained.

The above and other objects, features, and advantages of the present invention will become more apparent from the following description when taken in conjunction with the accompanying drawings, in which a preferred embodiment of the present invention is shown by way of illustrative example.

### BRIEF DESCRIPTION OF THE DRAWINGS

FIG. 1 is a schematic system diagram of a methane synthesis system;

FIG. 2 is a longitudinal cross-sectional view of principal components of a methane synthesis device in which a catalyst for methane synthesis according to the present embodiment is used;

FIG. 3 is a schematic diagram showing the structure of layered double hydroxides;

FIG. 4 is a schematic flowchart of a method of manufacturing a catalyst for methane synthesis according to the present embodiment;

FIG. 5 shows X-ray diffraction profiles of a generated product obtained by the procedures of Examples 1 and 2 and Comparative Examples 1 to 3;

FIG. 6 is a chart showing a comparison between Examples 1 and 2 and Comparative Examples 1 to 12, and catalyst names;

FIG. 7 is a chart showing a BET surface area, a pore volume, and a pore diameter of the respective catalysts of Examples 1 and 2 and Comparative Examples 4, 5, and 8 to 12;

FIG. 8 is a graph showing a CO conversion ratio when methane synthesis was carried out using the respective catalysts of Examples 1 and 2 and Comparative Examples 1 to 12;

FIG. 9 is a graph showing a methane selectivity when methane synthesis was carried out using the respective catalysts of Examples 1 and 2 and Comparative Examples 1 to 12;

FIG. 10 is a graph showing a methane generation ratio when methane synthesis was carried out using the respective catalysts of Examples 1 and 2 and Comparative Examples 1 to 12;

FIG. 11 is a graph showing an amount of methane generated per unit weight of the catalyst when methane synthesis was carried out using the respective catalysts of Examples 1 and 2 and Comparative Examples 1 to 12;

FIG. 12 is a graph showing a CO conversion ratio when methane synthesis was carried out using the respective catalysts of Examples 1 and 2 and Comparative Examples 5 and 9 while changing a supply flow rate of a mixed gas of carbon monoxide and hydrogen;

FIG. 13 is a graph showing a methane generation ratio when methane synthesis was carried out using the respective catalysts of Examples 1 and 2 and Comparative Examples 5 and 9 while changing the supply flow rate of the mixed gas of carbon monoxide and hydrogen;

FIG. 14 is a graph showing a CO conversion ratio when methane synthesis was carried out using the respective catalysts of Examples 1 and 2 and Comparative Example 5 while adding moisture to the mixed gas of carbon monoxide and hydrogen; and

FIG. 15 is a graph showing a methane generation ratio when methane synthesis was carried out using the respective catalysts of Examples 1 and 2 and Comparative Example 5 while adding moisture to the mixed gas of carbon monoxide and hydrogen.

### DETAILED DESCRIPTION OF THE INVENTION

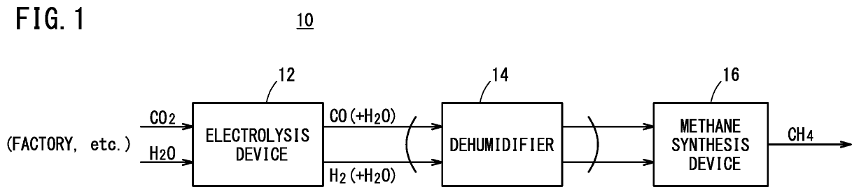

FIG. 1 is a schematic system diagram of a methane synthesis system 10 that obtains methane from carbon dioxide and water. The methane synthesis system 10 is equipped with an electrolysis device 12, a dehumidifier 14, and a methane synthesis device 16. As will be discussed later, it is also possible to eliminate the dehumidifier 14.

The electrolysis device 12 is a device that electrolyzes carbon dioxide and water together. As the carbon dioxide and water, for example, emissions from a factory, a garbage disposal site, or the like can be used. As the carbon dioxide, recovered carbon dioxide within the atmosphere may be used. It is also possible to condense water vapor and thereby obtain the water.

By the electrolysis of carbon dioxide and water (co-electrolysis), carbon monoxide and hydrogen are generated. Since water (liquid water) is used as the hydrogen source, the carbon monoxide and the hydrogen are wet gases. The dehumidifier 14 removes moisture from the carbon monoxide and the hydrogen. However, as discussed previously, the dehumidifier 14 may be eliminated.

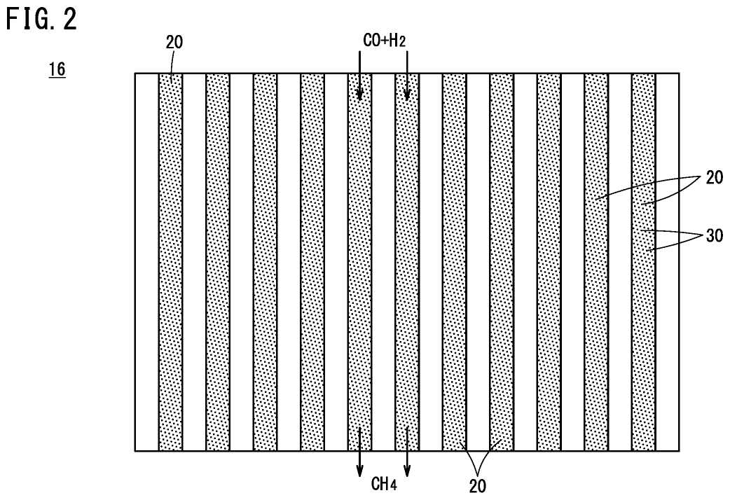

The methane synthesis device 16 is a reactor for synthesizing methane from carbon monoxide and hydrogen. In the interior of the methane synthesis device 16, a plurality of column tubes 20 are provided as shown in FIG. 2. A powdered methane synthesis catalyst 30 is provided in each of the column tubes 20. Hereinafter, the methane synthesis catalyst 30 is also simply referred to as a “catalyst 30”.

In the present embodiment, the catalyst 30 is made up from layered double hydroxides (LDHs). More specifically, the general formula of the catalyst 30 is represented by the following formula (1).

[M²⁺₁₋ₓM³⁺ₓ(OH)₂]^{x+}[Aⁿ⁻_{x/n}·yH₂O]^{x-} (1)

In this instance, M²⁺ is a divalent metal ion and M³⁺ is a trivalent metal ion. According to the present embodiment, M²⁺ is Ni²⁺, and M³⁺ is Al³⁺ or Cr³⁺. Aⁿ⁻ is an anion, which in the present embodiment is CO₃²⁻. The term x lies within a range of 0.19 to 0.34 (0.19≤x≤0.34), and y is 0 or a positive integer. More specifically, it is also possible for the layered double hydroxides to adopt a structure that does not contain water between the layers. Further, since dehydration and rehydration reactions occur reversibly depending on the temperature, y is a variable.

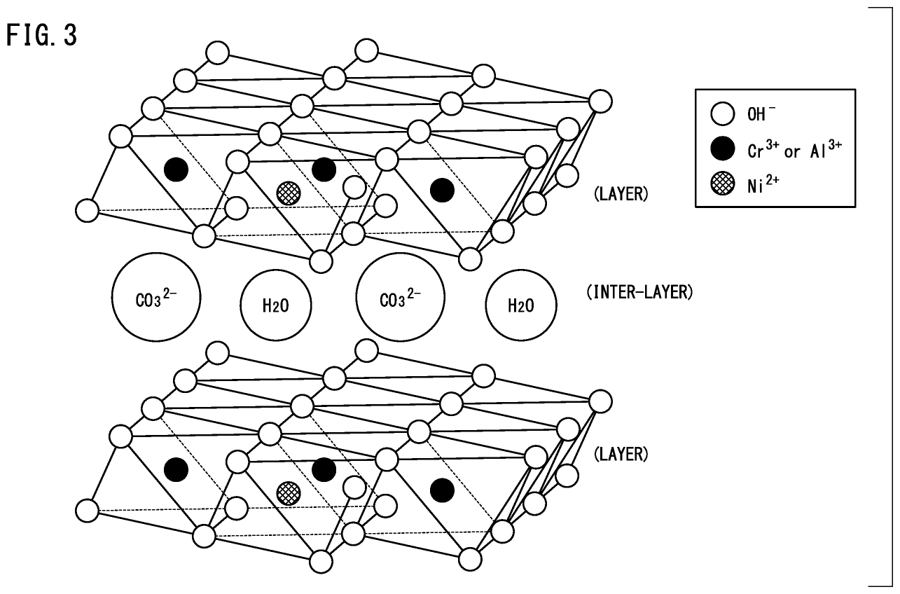

The structure of the layered double hydroxides is shown schematically in FIG. 3. The layered double hydroxides include a plurality of layers containing M²⁺, M³⁺, and OH⁻. Anions (Aⁿ⁻) and water are interposed between two individual ones of the layers. As can be understood from this feature, the layered double hydroxides are an interlayer compound.

A general catalyst for methane synthesis is filled into the column tubes 20 in a state of being supported on a carrier. In contrast thereto, according to the present embodiment, it is possible for the powdered catalyst 30 to be filled solely into the column tubes 20. In other words, there is no particular need for the catalyst 30 to be supported on a carrier.

As will be described later, in the case that the catalyst 30 is used, a large amount of carbon monoxide is converted into methane at a high rate. In other words, the catalyst 30 is superior in terms of its CO conversion ratio. Furthermore, in the case that the catalyst 30 is used, the amount of generated products (by-products) other than methane is small. More specifically, owing to the catalyst 30, methane is preferentially generated. In this manner, the catalyst 30 is also superior in terms of its methane selectivity.

Furthermore, as can be understood from the above-described formula (1), the catalyst 30 can retain water in the form of a hydrate within its molecules. Therefore, the catalyst 30 is chemically stable with respect to water. More specifically, the catalyst 30 exhibits superior resistance to water. Accordingly, even in the case that carbon dioxide and hydrogen are supplied to the methane synthesis device 16 as wet gases containing moisture, deterioration of the catalyst 30 in the column tubes 20 is suppressed over a prolonged period of time.

For such a reason, according to the present embodiment, it is possible to eliminate the dehumidifier 14 from the methane synthesis system 10. In this case, the configuration of the methane synthesis system 10 is simplified. Further, it is possible to reduce the cost of investment in equipment. In addition, since it is possible to eliminate incidental equipment and the like provided in the dehumidifier 14, the running cost when continuously operating the methane synthesis system 10 is reduced.

Based on the above, according to the present embodiment, it is possible to reduce the manufacturing cost of methane.

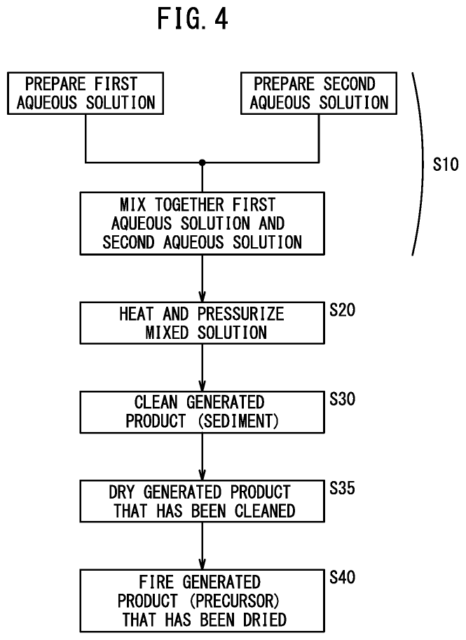

Next, with reference to the schematic flowchart shown in FIG. 4, a description will be given concerning a method of manufacturing the catalyst 30. This manufacturing method includes a mixing step S10, a heating step S20, a cleaning step S30, and a firing step S40. A drying step S35 is executed between the cleaning step S30 and the firing step S40.

First, in the mixing step S10, an operator prepares a first aqueous solution and a second aqueous solution. In the case that the trivalent metal ion is Cr³⁺, the first aqueous solution is prepared by dissolving a nickel compound serving as a nickel source, and a chromium compound serving as a chromium source in the same deionized water. In this instance, nickel nitrate is selected as the nickel compound. Moreover, the nickel nitrate may be in the form of a hydrate. A typical example of a hydrate of nickel nitrate is a hexahydrate. The chemical formula for the hexahydrate of nickel nitrate is shown below.

Ni(NO₃)₂·6H₂O

Further, chromium nitrate is selected as the chromium compound. The chromium nitrate may be in the form of a hydrate. A typical example of a hydrate of chromium nitrate is a nonahydrate. The chemical formula for the nonahydrate of chromium nitrate is shown below.

Cr(NO₃)₃·9H₂O

In the case that the value of x in the above-described formula (1) is 0.25, nickel nitrate and chromium nitrate are added to deionized water in an amount such that the molar ratio of nickel and chromium is Ni:Cr=3:1.

In the case that the trivalent metal ion is Al³, the first aqueous solution is prepared by dissolving a nickel compound serving as a nickel source, and an aluminum compound serving as an aluminum source in the same deionized water. In this instance, aluminum nitrate is selected as the aluminum compound. The aluminum nitrate may be in the form of a hydrate. A typical example of a hydrate of aluminum nitrate is a nonahydrate. The chemical formula for the nonahydrate of aluminum nitrate is shown below.

Al(NO₃)₃·9H₂O

In the case that the value of x in the above-described formula (1) is 0.25, nickel nitrate and aluminum nitrate are added to deionized water in an amount such that the molar ratio of nickel and aluminum is Ni:Al=3:1.

The first aqueous solution which is prepared in the manner described above is preferably stirred. In the case of stirring the first aqueous solution, there is no particular necessity to heat the first aqueous solution. Further, such stirring can be carried out under atmospheric conditions.

Separately from the above, the operator prepares the second aqueous solution. The second aqueous solution is prepared by dissolving a carbonate compound, which serves as a source of carbonic acid, in deionized water. In this instance, urea is selected as the carbonate compound. The chemical formula for urea is shown below.

CO(NH₂)₂

The second aqueous solution is preferably stirred. In the case of stirring the second aqueous solution, there is no particular necessity to heat the second aqueous solution. Further, such stirring can be carried out under atmospheric conditions.

Thereafter, the first aqueous solution and the second aqueous solution are mixed together. Thus, a mixed solution is obtained. The mixed solution may be stirred. In this case as well, there is no particular necessity to heat the mixed solution. Further, such stirring can be carried out under atmospheric conditions.

Next, the heating step S20 is executed. In the heating step S20, the mixed solution obtained in the mixing step S10 is heated. According to the present embodiment, in the heating step S20, pressure is applied to the mixed solution. Specifically, the mixed solution is heated while pressure is applied thereto. More specifically, the mixed solution is sealed, for example, in an autoclave. In this state, the autoclave is heated to a predetermined temperature. Preferably, the heating rate at this time is moderate. A suitable heating rate, for example, lies within a range of 1 to 5° C. per minute.

The pressure inside the autoclave is on the order of 5 to 8 atm, for example. In accordance with this feature, the mixed solution is pressurized. After having reached the predetermined temperature, the temperature of the autoclave is maintained at the predetermined temperature. Preferably, the predetermined temperature lies within a range of 100 to 150° C., and in this case, a preferred maintenance time period lies within a range of 10 to 20 hours. During this period, it is not necessary to stir the mixed solution.

A hydrothermal reaction proceeds in the mixed solution that has become pressurized and heated to a high temperature. A generated product is generated by such a hydrothermal reaction, and precipitates within the mixed solution. Since the mixed solution is not subjected to stirring, the hydrothermal reaction proceeds statically. By filtering the mixed solution in which the generated product (a sediment) is contained, the generated product is separated from the mixed solution.

Next, the cleaning step S30 is executed. Specifically, the operator introduces the generated product and a cleaning liquid into a separation tank of a centrifugal separator, and thereafter, causes the separation tank to rotate. The cleaning liquid is discharged to the exterior of the separation tank. On the other hand, the generated product remains on the inner wall of the separation tank. Moreover, a typical example of the cleaning liquid is deionized water. A mixture of the deionized water and ethanol can also be used as the cleaning liquid.

In this instance, the operator measures the pH of the cleaning liquid that is discharged from the separation tank. In the case that the pH of the cleaning liquid is alkaline, the operator cleans the generated product with fresh cleaning liquid. The operator repeats this cycle until the cleaning liquid that is discharged from the separation tank exhibits neutrality. At the point in time when the cleaning liquid exhibits neutrality, the cleaning step S30 (cleaning of the generated product) comes to an end.

Next, preferably, the drying step S35 is executed. Specifically, the generated product that has been cleaned is dried at a temperature of less than or equal to 100° C. Since the drying temperature is comparatively low, even if the generated product is dried under atmospheric conditions, the generated product is prevented from chemically combining with oxygen within the atmosphere. More specifically, in the drying step, a situation is avoided in which the generated product becomes oxidized. Drying is continued on the order of 10 hours to 1 day, for example. At this point in time, the generated product is transformed into a precursor of double layered hydroxides.

Next, the firing step S40 is executed. In the firing step S40, the precursor, which was obtained through the steps up until the drying step S35, is fired at a predetermined temperature in a firing furnace. Moreover, preferably, the heating rate in the firing furnace is moderate. A suitable heating rate, for example, lies within a range of 1 to 5° C. per minute. Also, a suitable firing temperature lies within a range of 400 to 700° C. The firing temperature is maintained for a period of 3 to 10 hours, for example.

In this instance, when firing is carried out under atmospheric conditions in which oxygen is present, a concern arises in that oxidation of the precursor will take place. In this case, the layered double hydroxides cannot be obtained. In order to avoid this situation, it is preferable to create an inert atmosphere inside the firing furnace. More specifically, an inert gas is passed through the firing furnace, and firing is performed in this state. A preferred specific example of the inert gas is nitrogen gas. Since nitrogen gas tends to be inexpensive, an increase in the manufacturing cost of the layered double hydroxides (the catalyst 30) can be avoided.

Due to being fired, the precursor transforms into powdered layered double hydroxides. More specifically, the catalyst 30 is obtained in the form of a powder. At this point in time, the particle size of the layered double hydroxides is smaller than the particle size of layered double hydroxides obtained by a coprecipitation method. Further, although the layered double hydroxides obtained by such a coprecipitation method have a large variation in particle size, the particle size of the layered double hydroxides obtained in the manner described above is comparatively uniform. In other words, the particle size distribution width thereof is narrow.

If necessary, the catalyst 30 is classified using a sieve. By being classified in this manner, the particle size of the catalyst 30 can be made more uniform.

### EXAMPLES

### Example 1

The first aqueous solution was prepared by dissolving 2.18 g of Ni(NO₃)₂·6H₂O and 0.94 g of Al(NO₃)₃·9H₂O in 60 ml of deionized water. The first aqueous solution was stirred at room temperature for 30 minutes at a stirring speed of 300 rpm. Apart therefrom, the second aqueous solution was prepared by dissolving 6 g of urea in 60 ml of deionized water. Thereafter, the second aqueous solution was stirred at room temperature for 30 minutes at a stirring speed of 300 rpm. Furthermore, the entire amount of the first aqueous solution and the entire amount of the second aqueous solution were mixed together and stirred at room temperature for 5 minutes at a stirring speed of 300 rpm. Thus, the mixed solution was prepared.

Next, the mixed solution was sealed in a stainless steel autoclave. Via the autoclave, the mixed solution was heated to 120° C. at a heating rate of 3° C. per minute. Thereafter, the autoclave was maintained at 120° C. for a period of 12 hours. In the foregoing process, stirring was not performed.

When the mixed solution was visually observed after cooling, it was confirmed that a sediment had been generated within the mixed solution. Next, the mixed solution was filtered, and the solvent and the sediment were separated. After cleaning the sediment with 200 ml of deionized water, cleaning water containing the sediment was introduced into a separation tank of a centrifugal separator. By causing the separation tank to rotate, the sediment was separated from the cleaning water. The pH of the cleaning water discharged from the separation tank was measured. The above-described cleaning operation was repeated until the cleaning water discharged from the separation tank exhibited neutrality.

The sediment was then subjected to drying in air at 60 to 80° C. The drying time period was 12 hours. In accordance therewith, the precursor of the layered double hydroxides was obtained.

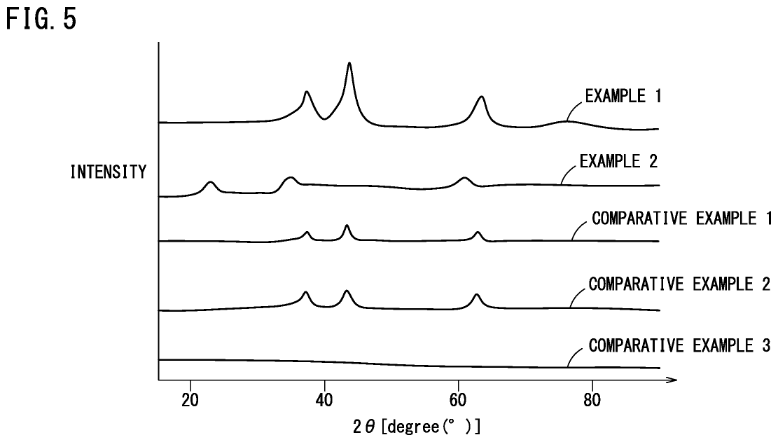

Next, the precursor was introduced into the firing furnace and placed in an N₂ atmosphere inside the firing furnace, and the temperature was raised to 500° C. at a heating rate of 2° C. per minute. Thereafter, the firing furnace was maintained at 500° C. for a period of 4 hours. Thus, the precursor was subjected to a firing process. X-ray diffraction profiles of the fired product that was obtained are shown in FIG. 5. From the fact that a metal oxide peak does not appear in FIG. 5, it can be understood that layered double hydroxides (LDHs) containing Ni²⁺, Al³⁺, and CO₃²⁻ were obtained. Such layered double hydroxides are referred to as Example 1. The obtained layered double hydroxides were classified using a sieve.

Concerning Example 1, a theoretical chemical formula of the layered double hydroxides is shown below.

[Ni²⁺_{0.75}Al³⁺_{0.25}(OH)₂]^{0.25+}[CO₃²⁻_{0.25/2}]^{0.25-}

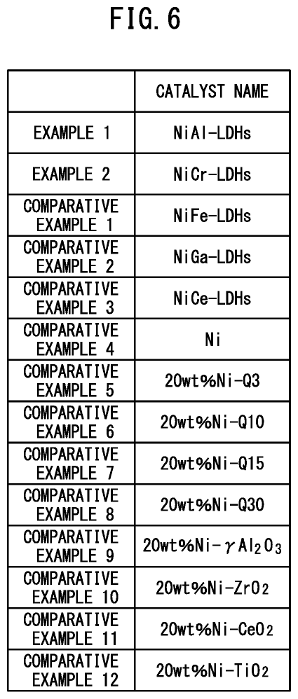

In FIG. 6, Example 1 is expressed as “NiAl-LDHs”.

### Example 2

Layered double hydroxides containing Cr³⁺ instead of Al³ were obtained in the same manner as in Example 1, except that 1.0 g of Cr(NO₃)₃·9H₂O was used. More specifically, the layered double hydroxides contain Ni²⁺, Cr³⁺, and CO₃²⁻. Such layered double hydroxides are referred to as Example 2. Concerning Example 2, a theoretical chemical formula of the layered double hydroxides is shown below.

[Ni²⁺_{0.75}Cr³⁺_{0.25}(OH)₂]^{0.25+}[CO₃²⁻_{0.25/2}]^{0.25-}

Further, X-ray diffraction profiles of the LDHs of Example 2 are also shown in FIG. 5. As can be understood from FIG. 5, a metal oxide peak also does not appear in the X-ray diffraction profiles of Example 2. Moreover, in FIG. 6, Example 2 is expressed as “NiCr-LDHs”.

### Comparative Examples 1 to 3

Layered double hydroxides containing either one of Fe³⁺, Ga³⁺, or Ce³⁺ instead of Al³⁺ were obtained in the same manner as in Example 1, except that an iron nitrate, gallium nitrate, or cerium nitrate was used. Such layered double hydroxides are referred to respectively as Comparative Examples 1 to 3. The X-ray diffraction profiles of the LDHs of Comparative Examples 1 to 3 are also shown in FIG. 5. Moreover, in FIG. 6, Comparative Examples 1 to 3 are expressed respectively as “NiFe-LDHs”, “NiGa-LDHs”, or “NiCe-LDHs”.

### Comparative Example 4

Ni was selected as the catalyst. This is referred to as Comparative Example 4.

### Comparative Examples 5 to 8

Catalysts were obtained by supporting Ni on a silica-based carrier. The supported amount was 20% by weight (wt), and Q3, Q10, Q15, or Q30 manufactured by Fuji Silysia Chemical, Ltd. was used as the carrier. It should be noted that Q3, Q10, Q15, and Q30 are product names. Such catalysts are referred to respectively as Comparative Examples 5 to 8. In FIG. 6, Comparative Examples 5 to 8 are expressed respectively as “20 wt % Ni-Q3”, “20 wt % Ni-Q10”, “20 wt % Ni-Q15”, or “20 wt % Ni-Q30”.

### Comparative Examples 9 to 12

Catalysts were obtained by supporting Ni on a carrier in the same manner as in Comparative Examples 5 to 8, except that γ-alumina, zirconia, ceria, or titania was used as the carrier. Such catalysts are referred to as Comparative Examples 9 to 12. In FIG. 6, Comparative Examples 9 to 12 are expressed respectively as “20 wt % Ni-γAl₂O₃”, “20 wt % Ni—ZrO₂”, “20 wt % Ni—CeO₂”, or “20 wt % Ni—TiO₂”.

In FIG. 6, a comparison between Examples 1 and 2 and Comparative Examples 1 to 12, and catalyst names are shown.

### [Characterization in Relation to Pores]

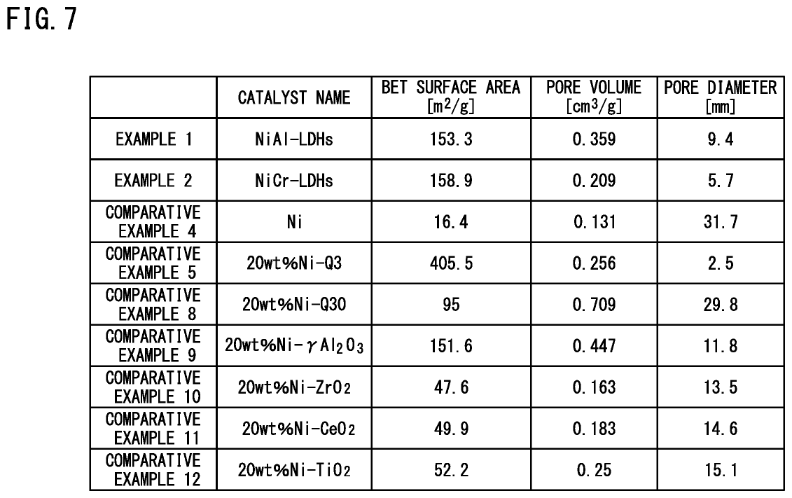

The BET surface areas of the catalysts of Example 1, Example 2, Comparative Example 4, Comparative Example 5, Comparative Example 8, Comparative Example 9, Comparative Example 10, Comparative Example 11, and Comparative Example 12 were measured. Further, the pore volumes and the pore diameters thereof were evaluated. The results of this measurement are shown together in FIG. 7.

### [CO Conversion Ratio]

The column tubes were filled with each of the catalysts of Examples 1 and 2 or Comparative Examples 1 to 12, and the methane synthesis device was assembled. After the temperature of the methane synthesis device was raised to 300° C., a mixed gas of carbon monoxide and hydrogen was introduced into the column tubes. The flow rate of the mixed gas was 20 ml per minute. Thereafter, the exhaust gas discharged from the column tubes was sampled, and the exhaust gas was analyzed using a gas chromatograph. Specifically, components of chemical substances other than moisture contained in the exhaust gas, and the composition ratio of the components were determined.

As a result, it was determined that, in the case that the catalyst of Example 1 or the catalyst of Example 2 was used, the exhaust gas did not contain CO. More specifically, in these cases, the entire amount of CO that was introduced into the column tubes was converted into other chemical substances. In other words, concerning the CO conversion ratio in the catalyst of Example 1 or the catalyst of Example 2, in both of such instances, the CO conversion ratio was 100%.

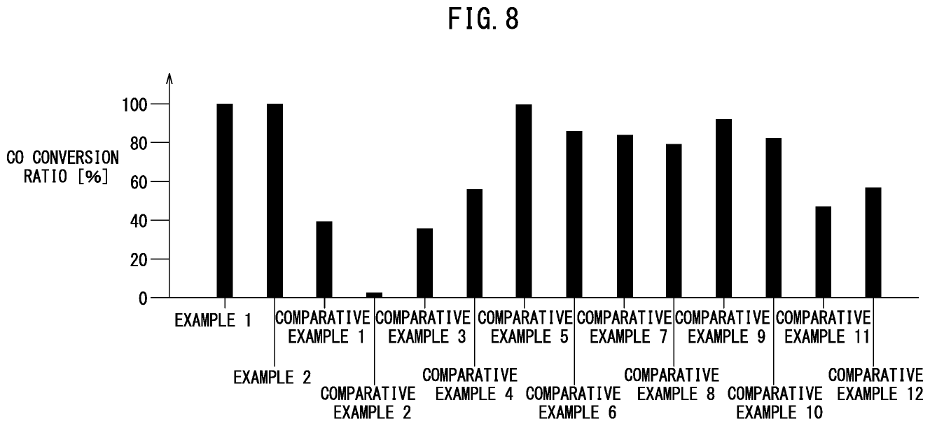

Similarly, concerning the catalysts of Comparative Examples 1 to 12 as well, whether or not the exhaust gas contained carbon monoxide was investigated, and in the case that the exhaust gas contained carbon monoxide, the composition ratio thereof was determined. The CO conversion ratio was determined based on the amount of carbon monoxide supplied to the column tubes, and the amount of carbon monoxide contained within the exhaust gas. The results of such a determination are shown in FIG. 8. In FIG. 8, the CO conversion ratios in Exemplary 1 and Example 2 are also shown. Referring to FIG. 8, it can be understood that the catalyst of Example 1 and the catalyst of Example 2 exhibit a superior CO conversion ratio.

### [Methane Selectivity]

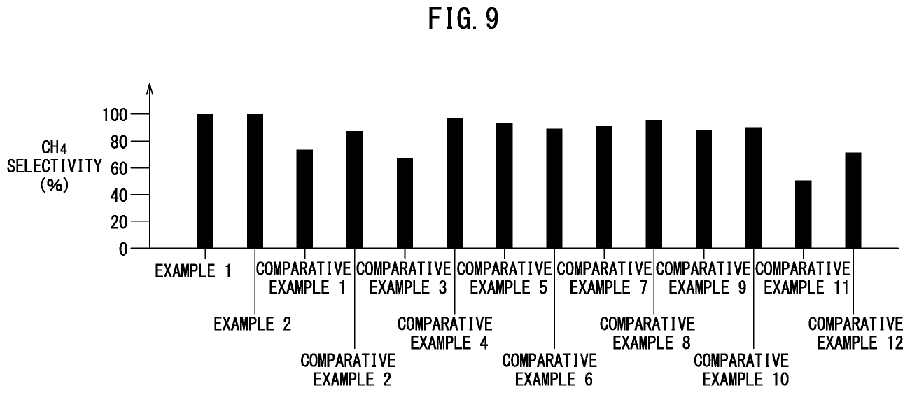

In the foregoing analysis, the composition ratio of methane was determined from the amount of methane with respect to the total amount of the exhaust gas. The composition ratio is shown and referred to as a methane selectivity in FIG. 9. The higher the value of the methane selectivity, the smaller the amount of by-products that are generated. More specifically, a methane generating reaction proceeds preferentially over the generating reactions of other chemical substances. The methane selectivity of the catalyst of Example 1 and the catalyst of Example 2 was 99.8%, which was higher than each of the catalysts of Comparative Examples 1 to 12.

### [Methane Generation Ratio]

Using the CO conversion ratio and the methane selectivity described above, the methane generation ratio was determined based on the following formula.

Methane Generation Ratio=CO Conversion Ratio×Methane Selectivity×0.01

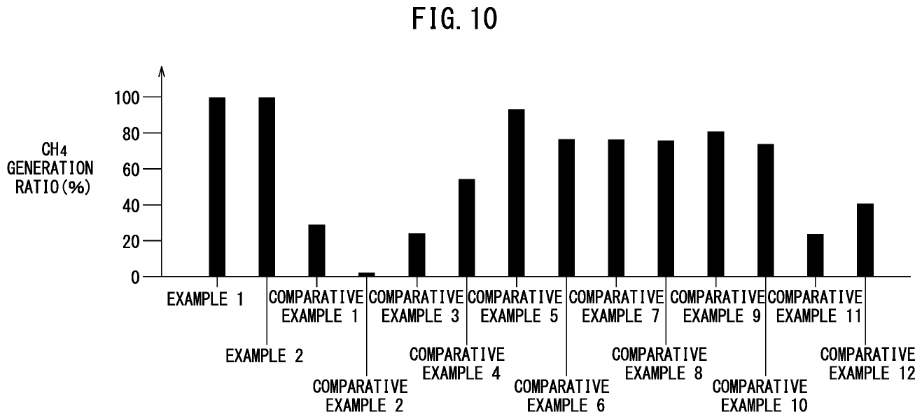

The methane generation ratios of the catalysts of Example 1 and Example 2, and Comparative Examples 1 to 12 are shown in FIG. 10. With reference to FIG. 10, it can be understood that, in the case that the catalyst of Example 1 and the catalyst of Example 2 are used, almost the entire amount of the carbon monoxide introduced into the column tubes was converted into methane. In contrast thereto, in the case that each of the catalysts of Comparative Examples 1 to 4 and 6 to 12 was used, the methane generation ratio was less than or equal to 85%. Moreover, it should be noted that the methane generation ratio in the case of using the catalyst of Comparative Example 5 was 93%.

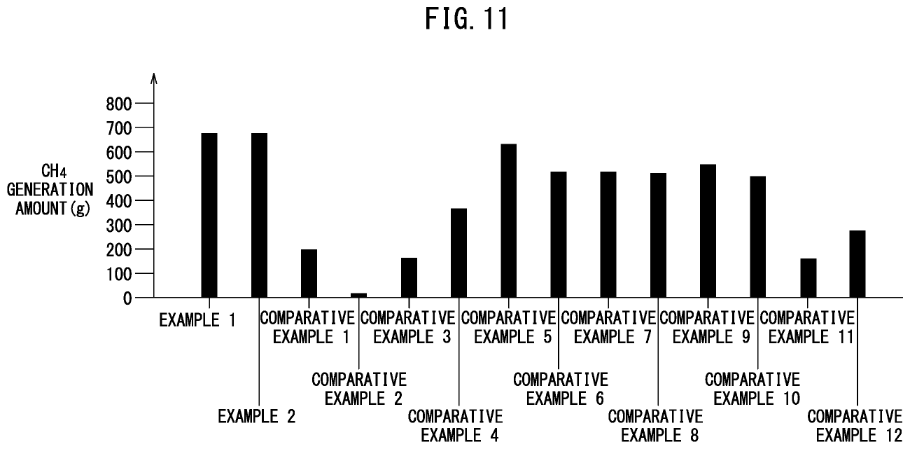

Further, the amount of methane generated per unit weight of the catalyst, and the amount of methane generated per unit time were calculated by dividing the amount of methane generated in the above-described methane synthesis by the mass of the catalyst and the time period. More specifically, due to the conversion, the amount of the methane generated per hour using 1 kg of catalyst was determined. The results of this determination are shown in FIG. 11.

From the above results, it is clear that, by using the catalyst of Example 1 (NiAl-LDHs) or the catalyst of Example 2 (NiCr-LDHs), carbon monoxide is converted into methane at a high rate. Accordingly, by using the catalyst of Example 1 and the catalyst of Example 2, carbon dioxide, which is a source of carbon monoxide, can be effectively consumed.

### [Evaluation of CO Processing Ability of Catalysts]

Concerning each of the catalysts of Example 1 and Example 2, and each of the catalysts of Comparative Example 5 and Comparative Example 9, the CO processing ability in the case that a large flow rate of mixed gas was introduced into the column tubes was investigated. In this instance, the catalyst of Comparative Example 5 (20 wt % Ni-Q3) and the catalyst of Comparative Example 9 (20 wt % Ni-γAl₂O₃) are catalysts that exhibited a comparatively high methane generation ratio.

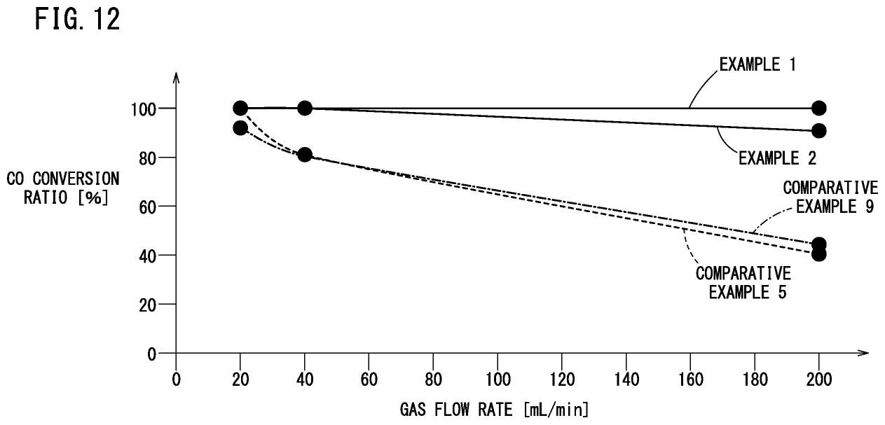

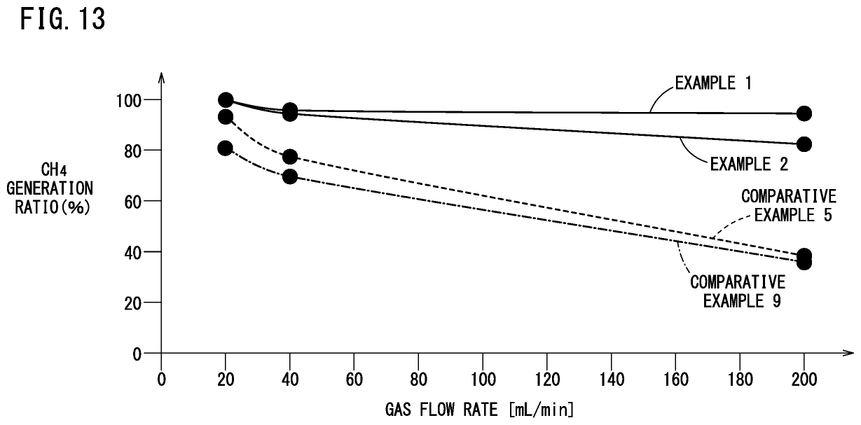

The flow rate of the carbon monoxide and the hydrogen introduced into the column tubes was changed to 40 ml per minute or 200 ml per minute. The CO conversion ratio and the methane generation ratio in each of such cases were calculated based on the results of the analysis. FIG. 12 shows the change in the CO conversion ratio, and FIG. 13 shows the change in the methane generation ratio. From FIG. 12 and FIG. 13, it can be understood that, in the case that the mixed gas is supplied at a large flow rate, the CO processing ability of the catalyst of Comparative Example 5 and the catalyst of Comparative Example 9 decreases. More specifically, unreacted carbon monoxide is mixed in the exhaust gas.

In contrast thereto, in the case that the catalyst of Example 1 and the catalyst of Example 2 are used, even in the case that the mixed gas is supplied at a large flow rate, the carbon monoxide is converted into methane at a high rate. More specifically, in accordance with the catalyst of Example 1 and the catalyst of Example 2, it is possible for a large amount of carbon monoxide to be processed in a short period of time.

### [Evaluation of Resistance to Water of Catalysts]

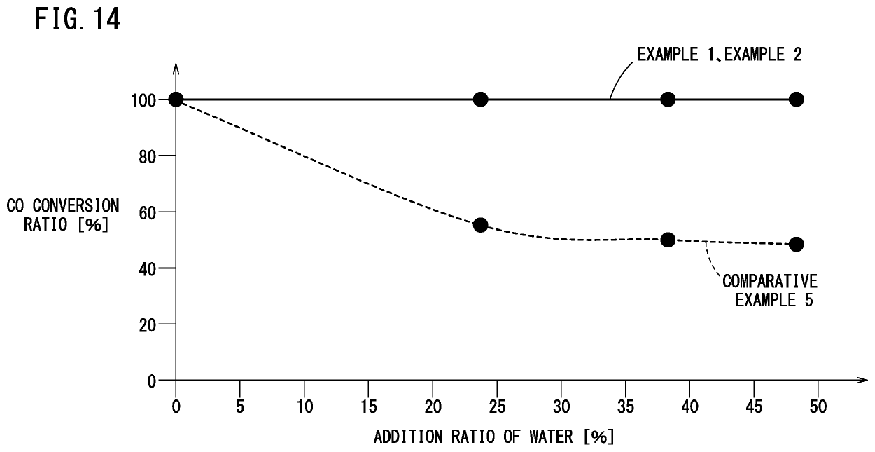

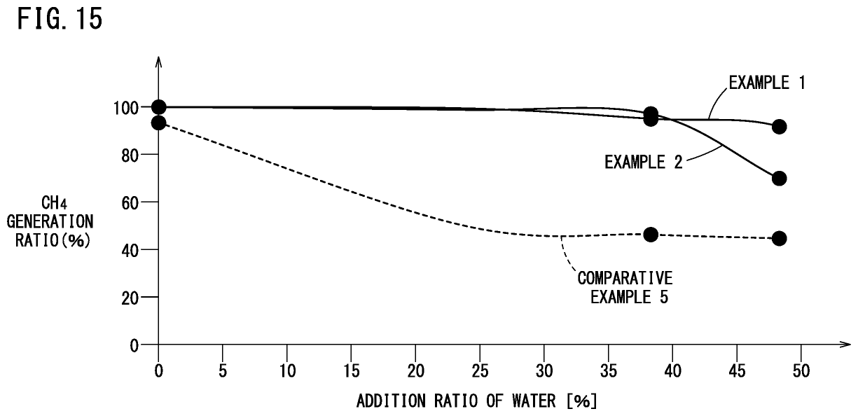

Using each of the catalysts of Example 1 and Example 2 and the catalyst of Comparative Example 5, a mixed gas to which moisture (water) was added was introduced into the column tubes. The addition ratio of the moisture was changed, and the change in the CO conversion ratio and the change in the methane generation ratio were determined. FIG. 14 shows the change in the CO conversion ratio with respect to the addition ratio of the moisture, and FIG. 15 shows the change in the methane generation ratio with respect to the addition ratio of the moisture.

From FIG. 14 and FIG. 15, it can be understood that, with the catalyst of Comparative Example 5, the CO conversion ratio and the methane generation ratio decrease significantly as the addition ratio of the moisture increases. In contrast thereto, in the case that the catalyst of Example 1 and the catalyst of Example 2 were used, even in the case that the addition ratio of the moisture is in excess of 30%, a sufficiently high CO conversion ratio and methane generation ratio are exhibited.

From this fact, it can be understood that, under the presence of the catalyst of Example 1 and the catalyst of Example 2, even in the case that a comparatively large amount of moisture is included in the carbon monoxide or the hydrogen, the carbon monoxide is converted into methane at a high rate. More specifically, the catalyst of Example 1 and the catalyst of Example 2 exhibit superior resistance to water.

Therefore, in the methane synthesis system in which the catalyst of Example 1 or the catalyst of Example 2 is used, it becomes unnecessary to provide a dehumidifier between the electrolysis device and the methane synthesis device. In this case, it is possible to simplify the methane synthesis system, and further, to reduce the cost of investment in equipment. Further, since it is possible to eliminate incidental equipment and the like provided in the dehumidifier, the running cost when continuously operating the methane synthesis system is reduced.

As noted previously, the present embodiment discloses the catalyst for methane synthesis (30) that is made up from layered double hydroxides represented by the following general formula (1), and that promotes the synthesis reaction for obtaining methane,

[M²⁺₁₋ₓM³⁺ₓ(OH)₂]^{x+}[Aⁿ⁻_{x/n}·yH₂O]^{x-} (1)

wherein, in formula (1), M²⁺ and Aⁿ⁻ are Ni²⁺ and CO₃², respectively, and M³⁺ is Al³⁺ or Cr³⁺, where 0.19≤x≤0.34, and y is 0 or a positive integer.

Typically, the catalyst for methane synthesis promotes a synthesis reaction for obtaining methane from carbon monoxide and hydrogen. More specifically, the present embodiment discloses the catalyst for methane synthesis that promotes a synthesis reaction for obtaining methane from carbon monoxide and hydrogen.

The catalyst for methane synthesis, which is made up from the layered double hydroxides containing Ni²⁺ as a divalent metal ion, Cr³⁺ or Al³⁺ as a trivalent metal ion, and CO₃ as an anion, exhibits excellent catalytic activity. More specifically, by using such a catalyst, it is possible to convert a large amount of carbon monoxide into methane within a short period of time. In addition, the generation of by-products (carbon dioxide or organic compounds) other than methane is suppressed. In this manner, such a catalyst is superior in terms of its ability to cause a large amount of carbon monoxide to participate in the reaction, and is also superior in terms of its ability to preferentially promote the synthesis reaction of methane. Furthermore, such a catalyst also exhibits superior resistance to water.

In addition, there is no particular need for the catalyst to be supported on a carrier. Therefore, it becomes unnecessary to select a carrier, and there is no need to carry out an operation for supporting the catalyst on such a carrier.

The present embodiment discloses the method of manufacturing the catalyst for methane synthesis that is made up from layered double hydroxides represented by the following general formula (1), and that promotes the synthesis reaction for obtaining methane,

[M²⁺₁₋ₓM³⁺ₓ(OH)₂]^{x+}[Aⁿ⁻_{x/n}·yH₂O] (1)

wherein, in formula (1), M²⁺ and Aⁿ⁻ are Ni²⁺ and CO₃²⁻, respectively, and M³⁺ is Al³⁺ or Cr³⁺, where 0.19≤x≤0.34, and y is 0 or a positive integer, the method including the mixing step (S10) of dissolving nickel nitrate, chromium nitrate or aluminum nitrate, and urea in water and thereby obtaining the mixed solution, the heating step (S20) of heating and pressurizing the mixed solution and thereby obtaining the generated product, the cleaning step (S30) of cleaning the generated product, and the firing step (S40) of firing the generated product that has been cleaned and thereby obtaining the layered double hydroxides.

Layered double hydroxides are generally obtained by a coprecipitation method. In this case, although powdered layered double hydroxides are obtained, the particle size thereof is large, and in addition, since the particle size thereof is non-uniform, the particle size distribution width thereof is wide. In contrast thereto, in the above-described manufacturing method, the hydrothermal reaction proceeds in the heating step. As a result, layered double hydroxides having a small particle size and a comparatively narrow particle size distribution width are obtained. As described above, by using such layered double hydroxides, the carbon monoxide can be converted into methane at a high rate.

The present embodiment discloses the method of manufacturing the catalyst for method synthesis, wherein water is used as the cleaning liquid in the cleaning step, and the cleaning of the generated product is repeated until the cleaning liquid exhibits neutrality.

In accordance with this feature, unreacted urea and the like can be removed from the generated product.

The present embodiment discloses the method of manufacturing the catalyst for methane synthesis, wherein the atmosphere in the firing step is an inert atmosphere.

In accordance with this feature, a situation is avoided in which the layered double hydroxides are oxidized during firing.

The present embodiment discloses the method of manufacturing the catalyst for methane synthesis, wherein, in the mixing step, the first aqueous solution of nickel nitrate and chromium nitrate or aluminum nitrate dissolved in water, and the second aqueous solution of urea dissolved in water are separately prepared, and the first aqueous solution and the second aqueous solution are mixed to thereby obtain the mixed solution.

In this case, it is easy to adjust the molar concentration of the divalent metal ion (Ni²⁺) and the molar concentration of the trivalent metal ion (Cr³⁺ or Al³⁺). Further, it is also easy to adjust the molar concentration of both of the metal ions, and the molar concentration of the anion (CO₃²⁻) obtained from the urea. Accordingly, it is possible to obtain the layered double hydroxides in which the molar ratio of Ni²⁺, the molar ratio of Cr³⁺ or Al³⁺, and the molar ratio of CO₃²⁻ are adjusted to desired molar ratios.

The present embodiment discloses the method of manufacturing the catalyst for methane synthesis, wherein, at the time of preparing the first aqueous solution and the second aqueous solution, the first aqueous solution and the second aqueous solution are separately stirred, and at the time of preparing the mixed solution, the mixed solution is stirred.

Due to being stirred, the nickel nitrate, and the chromium nitrate or the aluminum nitrate are sufficiently dissolved in the water. Further, in the first aqueous solution, the Ni²⁺ and the Cr³⁺ or the Al³⁺ are distributed substantially uniformly. The same is true concerning the urea and the CO₃²⁻ as well. Accordingly, in the heating step, the generated product can be obtained with a satisfactory yield.

Moreover, the present invention is not limited to the above-described disclosure, and various configurations can be adopted therein without departing from the essence and gist of the present invention.

1. A catalyst for methane synthesis that is made up from layered double hydroxides represented by a following general formula, and that promotes a synthesis reaction for obtaining methane,
[M²⁺₁₋ₓM³⁺ₓ(OH)₂]^{x+}[Aⁿ⁻_{x/n}·yH₂O]^{x-} (1)
wherein, in the general formula, M²⁺ and Aⁿ⁻ are Ni²⁺ and CO₃²⁻, respectively, and M³⁺ is Al³⁺ or Cr³⁺,
where 0.19≤x≤0.34, and y is 0 or a positive integer.

2. The catalyst for methane synthesis according to claim 1, wherein the catalyst promotes a synthesis reaction for obtaining methane from carbon monoxide and hydrogen.

3. A method of manufacturing a catalyst for methane synthesis that is made up from layered double hydroxides represented by a following general formula, and that promotes a synthesis reaction for obtaining methane,
[M²⁺₁₋ₓM³⁺ₓ(OH)₂]^{x+}[Aⁿ⁻_{x/n}·yH₂O]^{x-} (1)
wherein, in the general formula, M²⁺ and Aⁿ⁻ are Ni²⁺ and CO₃²⁻, respectively, and M³⁺ is Al³⁺ or Cr³⁺, where 0.19≤x≤0.34, and y is 0 or a positive integer,
the method comprising:
dissolving nickel nitrate, chromium nitrate or aluminum nitrate, and urea in water and thereby obtaining a mixed solution;
heating and pressurizing the mixed solution and thereby obtaining a generated product;
cleaning the generated product; and
firing the generated product that has been cleaned and thereby obtaining the layered double hydroxides.

4. The method of manufacturing the catalyst for methane synthesis according to claim 3, wherein water is used as a cleaning liquid in the cleaning of the generated product, and the cleaning of the generated product is repeated until the cleaning liquid exhibits neutrality.

5. The method of manufacturing the catalyst for methane synthesis according to claim 3, wherein an atmosphere in the firing of the generated product is an inert atmosphere.

6. The method of manufacturing the catalyst for methane synthesis according to claim 3, wherein, in the obtaining of the mixed solution, a first aqueous solution of nickel nitrate and chromium nitrate or aluminum nitrate dissolved in water, and a second aqueous solution of urea dissolved in water are separately prepared, and the first aqueous solution and the second aqueous solution are mixed to thereby obtain the mixed solution.

7. The method of manufacturing the catalyst for methane synthesis according to claim 6, wherein, at a time of preparing the first aqueous solution and the second aqueous solution, the first aqueous solution and the second aqueous solution are separately stirred, and at a time of preparing the mixed solution, the mixed solution is stirred.
